(19) United States
(12) Patent Application Publication
(10) Pub. No.: US 2017/0035480 A1
(43) Pub. Date: Feb. 9, 2017

(54) INTERNAL BONE FIXATION DEVICE

(71) Applicant: Human Origin Biotechnology Co., Ltd., New Taipei City (TW)

(72) Inventors: LIEN-GUO DAI, Taichung (TW); YA-HUI CHANG, Taichung (TW); SHIUAN-YUAN DAI, Taichung (TW); SHIUAN-TE DAI, Taichung (TW); SHIUAN-JEN DAI, Taichung (TW)

(73) Assignee: Human Origin Biotechnology Co., Ltd., New Taipei City (TW)

(21) Appl. No.: 15/227,981

(22) Filed: Aug. 4, 2016

(30) Foreign Application Priority Data
Aug. 5, 2015 (TW) 104125361

(51) Int. Cl. A61B 17/82

(52) U.S. Cl. CPC A61B 17/82; A61B 2017/00004

(57) ABSTRACT

Disclosed herein is a bone fixation device. The fixation device has a head portion having a conduit and a plurality of pawls formed therein along one side of the conduit; and an elongation strip having a hooked end and a plurality of ratchet teeth formed thereon the body of the strip. The head portion is connected to the non-hooked end of the elongation strip at an angle between 100-170 degrees, and the plurality of pawls of the head portion engage the plurality of teeth when the elongation strip is inserted into the conduit of the head portion to form a loop around the weaken or fractured bone so as to stabilize the weaken or fractured bone.

### CROSS-REFERENCE TO RELATED APPLICATIONS

This application claims foreign priority benefits under 35 U.S.C. §119(a)-(d) to Taiwan Patent Application No. 104125361 filed Aug. 5, 2015, which is hereby incorporated by reference in its entirety.

### BACKGROUND OF THE INVENTION

1. Field of the Invention

The embodiments disclosed herein relate to medical devices for use in repairing a weakened or fractured bone, and more particularly to internal bone fixation devices and methods for using the devices to repair a weakened or fractured bone.

2. Description of Related Art

Fracture repair is the process of rejoining and realigning the ends of broken bones. Fracture repair is required when there is a need for restoration of the normal position and function of the broken bone. Throughout the stages of fracture healing, the bones must be held firmly in the correct position and supported until it is strong enough to bear weight. In the event the fracture is not properly repaired, malalignment of the bone may occur, resulting in possible physical dysfunction of the bone or joint of that region of the body.

Currently there are several approaches to repair, strengthen and support a fractured bone. They include the use of bone fixation devices, such as wires, plates, rods, pins, nails, and screws to support the bone directly, and/or the addition of bone cement mixtures to a fractured bone. However, subsequent surgeries are required to remove these bone fixation devices after the fractured bones are healed.

Thus, there is a need in the art for an improved bone fixation device that repair, strengthen and support a fractured bone, the improved bone fixation device is easy to use, biodegradable, and causes minimal damage to the bone and supporting tissues.

### SUMMARY

Internal bone fixation devices and methods for using the devices to repair a weakened or fractured bone are disclosed herein. According to aspects illustrated herein, there is provided a device for repairing a fractured bone that includes a head portion having a conduit and a plurality of pawls formed therein along one side of the conduit; and an elongation strip having a hooked end and a plurality of ratchet teeth formed thereon along one side of the body of the strip. The head portion is connected to the non-hooked end of the elongation strip at an obtuse angle between 100-170 degrees, and the plurality of pawls of the head portion may engage the plurality of ratchet teeth when the elongation strip is inserted into the conduit of the head portion to form a loop around the weaken or facture bone.

According to some embodiments of the present disclosure, the head portion and the elongation strip of the bone fixation device are connected by forming a joined portion having an arc of 0.175 rad to 1.4 rad.

According to some embodiments of the present disclosure, the bone fixation device is composed by a bioabsorable material. Examples of bioabsorable material include, but are not limited to, alginate, biphasic calcum phosphate, calcium phosphate, calcium sulfate, chitosan, collagen, gelatin, hydroxyapatite (HA), polyalkylene esters, polyanhydrides, polyamide esters, polydioxanone, polyethylene glycol (PEG), polyactic acid (PLA) poly(lactic-co-glycolic acid) (PLGA), PLA-PLGA copolymer, poly(3-hydroxybutyrate-co-3-hydroxyvalerate), polyvinyl esters, polyvinyl alcohols, and tricalcium phosphate.

According to preferred embodiments of the present disclosure, the bone fixation device is an integral article.

The present bone fixation device may be used for the repair of bones that are weakened or fractured due to any of the bone diseases, which include, but are not limited to, osteoporosis, achondroplasia, bone cancer, fibrodysplasia ossificans progressiva, fibrous dysplasia, legg calve perthes disease, myeloma, osteogenesis imperfecta, osteomyelitis, osteopenia, osteoporosis, Paget's disease, scoliosis, and other similar diseases; or for the repair of bones that are weakened or fractured due to an injury, for example, a fall.

Examples of the weakened or fractured bone include, but are not limited to, metacarpal bone in the hand, the femur, tibia, fibula, humerus, ulna, radius, metatarsals, phalanx, phalanges, ribs, spine, vertebrae, clavicle and the like.

According to aspects illustrated herein, there is provided a method for repairing a weaken or fractured bone that includes providing a device for use in repairing the weaken or fractured bone, the device comprises in its structure, a head portion having a conduit and a plurality of pawls formed therein along one side of the conduit; and an elongation strip having a hooked end and a plurality of ratchet teeth formed thereon along one side of the body of the strip. The is head portion is connected to the non-hooked end of the elongation strip at an obtuse angle between 100-170 degrees, and the plurality of pawls of the head portion may engage the plurality of teeth when the elongation strip is inserted into the conduit of the head portion to form a loop around the weaken or facture bone.

According to some embodiments of the present disclosure, the head portion and the elongation strip of the bone fixation device are connected by forming a joined portion having an arc of 0.175 rad to 1.4 rad.

According to some embodiments of the present disclosure, the bone fixation device is composed by a bioabsorable material. Examples of bioabsorable material include, but are not limited to, alginate, biphasic calcum phosphate, calcium phosphate, calcium sulfate, chitosan, collagen, gelatin, hydroxyapatite (HA), polyalkylene esters, polyanhydrides, polyamide esters, polydioxanone, polyethylene glycol (PEG), polyactic acid (PLA) poly(lactic-co-glycolic acid) (PLGA), PLA-PLGA copolymer, poly(3-hydroxybutyrate-co-3-hydroxyvalerate), polyvinyl esters, polyvinyl alcohols, and tricalcium phosphate.

According to preferred embodiments of the present disclosure, the device is an integral article.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1 is a schematic drawing illustrating the bone fixation device 100 in accordance with one embodiment of the present disclosure;

FIG. 2A is a schematic drawing depicting the bone fixation device 100 of FIG. 1 forming a loop in accordance with one embodiment of the present disclosure;

FIG. 2B is a schematic drawing depicting the loop of FIG. 2A is tighten by engaging the plurality of teeth 122 with the plurality of pawls 154 in accordance with one embodiment of the present disclosure; and

FIG. 3 is a schematic drawing illustrating the bone fixation device 300 in accordance with another embodiment of the present disclosure.

### DETAILED DESCRIPTION OF THE INVENTION

The detailed description provided below in connection with the appended drawings is intended as a description of the present disclosure and is not intended to represent the only forms in which the present disclosure may be constructed or utilized.

1. Definition

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or io unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

The term “host,” “subject,” or “patient” is used interchangeably herein and refers to a mammal, in which the present internal bone fixation device may be applied therein to stabilize any weaken or fractured bone. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “host,” “subject,” or “patient” intended to refer to both the male and female gender unless one gender is specifically indicated. Accordingly, the term “host,” “subject,” or “patient” comprises any mammal which may benefit from the bone fixation device of the present disclosure. Examples of a “host,” “subject,” or “patient” include, but are not limited to, a human, rat, mouse, guinea pig, monkey, pig, goat, cow, horse, dog, cat, bird and fowl. In an exemplary embodiment, the patient is a human.

2. The Present Internal Bone Fixation Device

Medical devices and methods for repairing weakened or fractured bones are disclosed herein. The devices disclosed herein act as internal bone fixation devices for stabilize internal weaken or fractured bones without the need of trocars or guide wires that commonly required when conventional bone fixation devices are employed. Further, the present internal bone fixation devices are biodegradable, thus eliminating the need of a subsequent surgery to remove the device from its host after the fractured bones are healed.

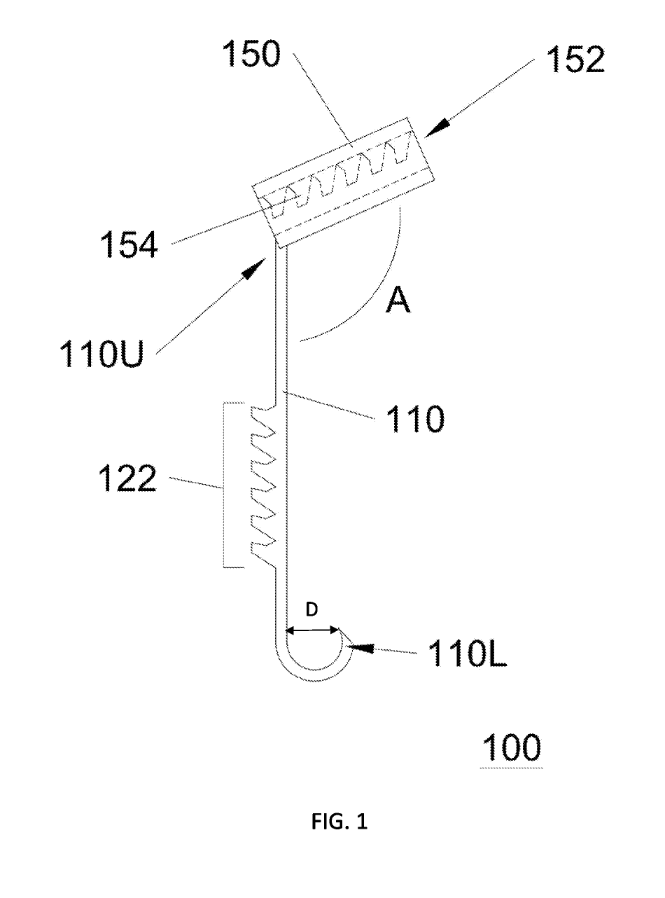

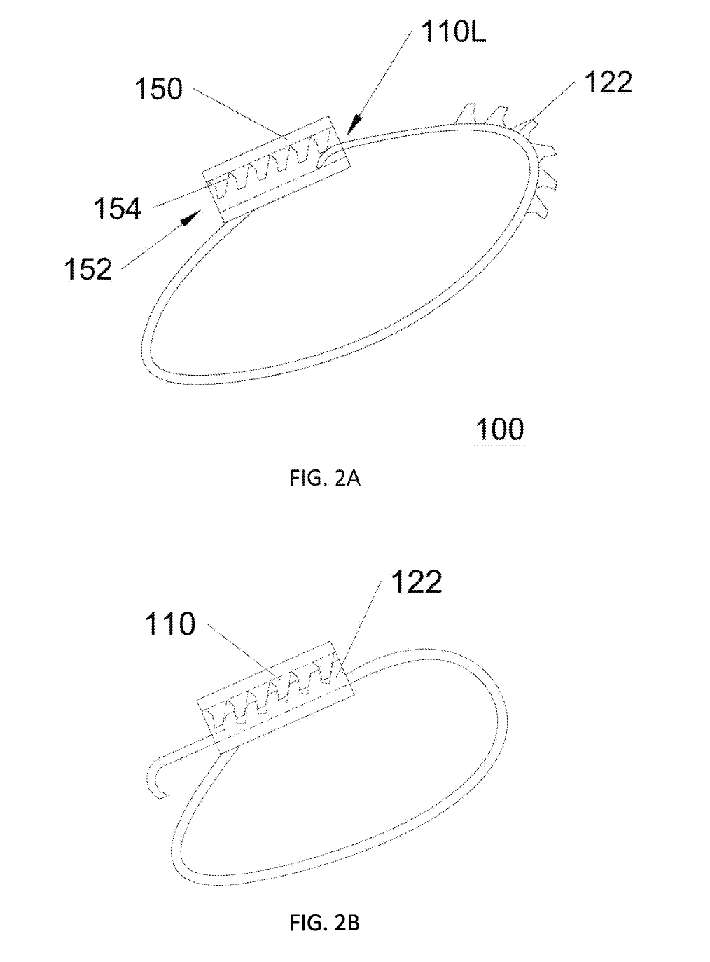

The main components of an embodiment of a device for repairing a weakened or fractured bone are depicted generally in FIG. 1 in conjunction with FIGS. 2A and 2B. The bone fixation device 100 includes a head portion 150, and an elongation strip 110 connected to the head portion 150 at an obtuse is angle (A). The head portion 150 comprises in its structure, a conduit 152, and a plurality of pawls 154 formed therein along one side of the conduit 152. The elongation strip 110 has an elongation body having a hooked end 110L, a non-hooked end 110U connected to the head portion 150, and a plurality of ratchet teeth 122 formed thereon along one side of the elongation body of the strip 110. It is to be noted that the tip of the hooked end 110L is sharpen enough to cut through muscles around a fractured bone, thus when the device 100 is in use, the elongation strip 110 can be inserted from the cut muscles and go around the fractured bone, then into the conduit 152 of the head portion 150 to form a loop around the fractured bone, the loop is then tighten by pulling the elongation strip 110 through the conduit 152 to engage the plurality of ratchet teeth 122 with the plurality of pawls 154, thereby stabilizing the fractured bone (FIGS. 2A and 2B). Engagement of the ratchet teeth 122 and the pawls 154 also prevents reverse movement of the strip 110 through the conduit 152 of the head portion 150. That way, the bone fixation device 100 is tighten around the weakened or fractured bone to hold the weakened or fractured bone in place.

According to embodiments of the present disclosure, the elongation strip 110 and the head portion 150 are connected at an obtuse angle (A) between 100-170 degrees, such as 100, 105, 110, 115, 120, 125, 130, 135, 140, 145, 150, 155, 160, 165, and 170 degrees; more preferably between 110-160 degrees, such as 110, 115, 120, 125, 130, 135, 140, 145, 150, 155, and 160 degrees. The obtuse angle between the elongation strip 110 and the head portion 150 allows the present bone fixation device 100 to comply with the shape or conformation of a bone surface when stabilizing a fractured bone, so that when the loop is tighten, such as by pulling the elongation strip 110 through the conduit 152 to engage the plurality of ratchet teeth 122 with the plurality of pawls 154, the stress generated therein can be distributed evenly to prevent the elongation strip 110 from being broken.

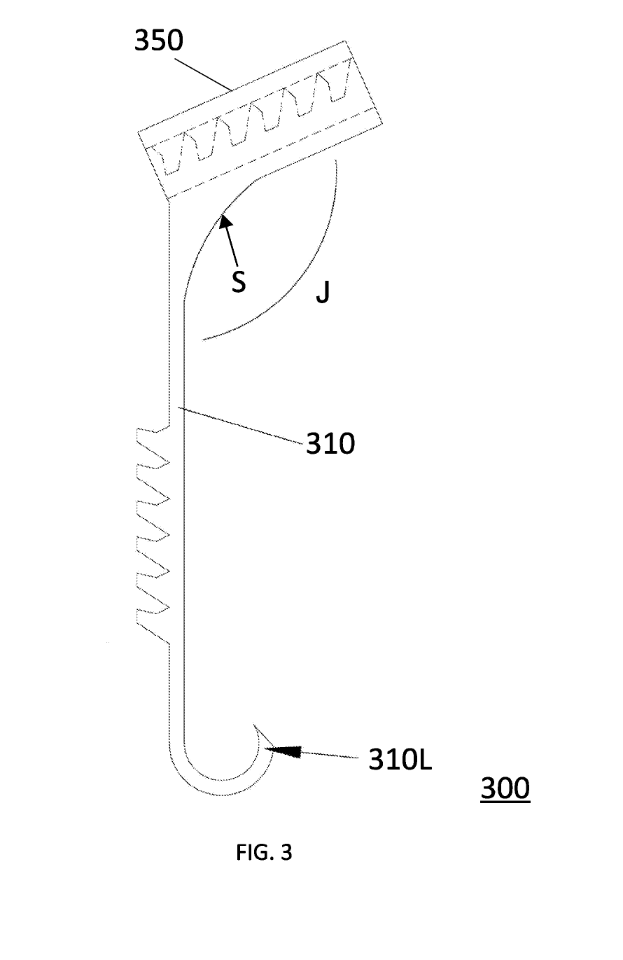

FIG. 3 depicts another embodiment of the present bone fixation device 300, in which the structure of the device 300 is relatively the same with the device 100 described above in FIGS. 1 and 2, except the connected portion between the elongation strip 310 and the head portion 350. In this embodiment, the elongation strip 310 and the head portion 350 are connected by forming a joined portion (J) having an arc of 0.175 to 1.4 rad (radian) at an obtuse angle (A) as defined above. The term “radian” refers to a unit for measuring angles. One radian is the angle made at the center of a circle by an arc whose length is equal to the radius of the circle, and is represented by the equation:

$$rad = \frac{π}{100} \times deg$$

By such design (i.e., the elongation strip 310 and the head portion 350 are connected by forming the joined potion (J) having an arc of 0.175 to 1.4 rad), the stress generated while stabilizing a fractured bone can be evenly distributed.

The present bone fixation device 100, 300 is preferably made by a material that is compatible and absorbable by its host (e.g., a human), such that the present device may be left inside the host after a fractured bone is stabilized, thereby eliminates the need of a subsequent surgery to remove the device therefrom. Examples of such material include, but are not limited to, alginate, biphasic calcum phosphate, calcium phosphate, calcium sulfate, chitosan, collagen, gelatin, hydroxyapatite (HA), polyalkylene esters, polyanhydrides, polyamide esters, polydioxanone, polyethylene glycol (PEG), polyactic acid (PLA) poly(lactic-co-glycolic acid) (PLGA), PLA-PLGA copolymer, poly(3-hydroxybutyrate-co-3-hydroxyvalerate), polyvinyl esters, polyvinyl alcohols, and tricalcium phosphate.

According to preferred embodiments of the present disclosure, the preset bone fixation device is formed into an integral article.

It should be noted that the present bone fixation device 100, 300 may come in various sizes and dimensions, and a physician may chose a suitable size of the present device according to the size and location of the weakened or fractured bone needs to be stabilized. According to some embodiments of the present disclosure the head portion 150, 350 of has a length of about 0.3-6.5 cm, such as 0,3, 0,4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4 and 6.5 cm. The elongation strip 110, 310 has a thickness of about 0.2-1 cm, such as 0.2, 0.3, 0.4, 0.5, 0.6, 0.7, 0.8, 0.9, and 1 cm, preferably about 0.2-0.5 cm, such as 0.2, 0.3, 0.4, and 0.5 cm; and a width of about 0.2-5 cm, such as 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 and 5 cm, preferably about 0.5-3 cm, such as 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0 cm; and a length of about 3-15 cm such as 3, 4, 5, 6, 7, 8, 9, 10, 11, 12, 13, 14, and 15 cm, preferably about 5-12 cm, such as 5, 6, 7, 8, 9, 10, 11, and 12 cm.

According to further embodiments of the present disclosure, the non-hooked end 110L, 310L of the elongation strip 110, 310 has a shape of a circle of 2/10- 9/10, such as a circle of 2/10, 3/10, 4/10, 5/10, 6/10, 7/10, 8/10 and 9/10; further, the tip of non-hooked end 110L, 310L is spaced apart from the elongation body by a distance (D) of about 0.5-7 cm, such as 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 and 7.0 cm.

The bone fixation device disclosed herein may be used for the repair of bones that are weakened or fractured due to any of the bone diseases, which include, but are not limited to, osteoporosis, achondroplasia, bone cancer, fibrodysplasia ossificans progressiva, fibrous dysplasia, legg calve perthes disease, myeloma, osteogenesis imperfecta, osteomyelitis, osteopenia, osteoporosis. Paget's disease, scoliosis. and other similar diseases. The bone fixation device disclosed herein may also be used for the repair of bones that are weakened or fractured due to an injury, for example, a fall.

Examples of the weakened or fractured bone include, but are not limited to, metacarpal bone in the hand, the femur, tibia, fibula, humerus, ulna, radius, metatarsals, phalanx, phalanges, ribs, spine, vertebrae, clavicle and other bones, which are still within the scope and spirit of the disclosed embodiments,

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A bone fixation device for stabilizing a weaken or fractured bone, comprising:
a head portion having a conduit and a plurality of pawls formed therein along one side of the conduit; and
an elongation strip having a hooked end and a plurality of ratchet teeth formed thereon along one side of the body of the strip;
wherein,
the head portion is connected to the non-hooked end of the elongation strip at an obtuse angle between 100-170 degrees, and
the plurality of pawls of the head portion engage the plurality of teeth when the elongation strip is inserted into the head portion to form a loop around the weaken or fractured bone so as to stabilize the weaken or fractured bone.

2. The bone fixation device of claim 1, wherein the head portion and the elongation strip are connected by forming a joined portion having an arc of 0.175 rad to 1.4 rad.

3. The bone fixation device of claim 1, wherein the device is composed by a bioabsorable material.

4. The bone fixation device of claim 3, wherein the bioabsorable material is selected from the group consisting of, alginate, biphasic calcum phosphate, calcium phosphate, calcium sulfate, chitosan, collagen, gelatin, hydroxyapatite (HA), polyalkylene esters, polyanhydrides, polyamide esters, polydioxanone, polyethylene glycol (PEG), polyactic acid (PLA) poly(lactic-co-glycolic acid) (PLGA), PLA-PLGA copolymer, poly(3-hydroxybutyrate-co-3-hydroxyvalerate), polyvinyl esters, polyvinyl alcohols, and tricalcium phosphate.

5. The bone fixation device of claim 1, wherein the device is an integral article.
